(19) United States
(12) Patent Application Publication
(10) Pub. No.: US 2020/0362418 A1
(43) Pub. Date: Nov. 19, 2020

(54) METHOD OF PREDICTION OF TUMOR-DERIVED NEO-PEPTIDE ANTIGENICITY AND/OR IMMUNOGENICITY USING MUTATIONAL SIGNATURE PATTERNS

(71) Applicant: CUREMATCH, INC., SAN DIEGO, CA (US)

(72) Inventors: Razelle Kurzrock, San Diego, CA (US); Igor Flint Tsigelny, San Diego, CA (US); Amelie Clemence Boichard, ECKBOLSHEIM (FR); Timothy Viet Pham, Anaheim, CA (US)

(73) Assignee: CUREMATCH, INC., SAN DIEGO, CA (US)

(21) Appl. No.: 16/652,857

(22) PCT Filed: Oct. 2, 2018
(86) PCT No.: PCT/US18/54042

§ 371 Date: Apr. 1, 2020

Related U.S. Application Data
(60) Provisional application No. 62/567,096, filed on Oct. 2, 2017.

(51) Int. Cl. C12Q 1/6886

(52) U.S. Cl. CPC C12Q 1/6886; C12Q 2600/106; C12Q 2600/156

(57) ABSTRACT

A method of prediction of response to immunotherapy for patients diagnosed with a proliferative, degenerative or inflammatory disease, is provided, the method comprising analysis of physicochemical properties of the set of neo-antigens produced by the injured tissue.

### CROSS-REFERENCE TO RELATED APPLICATIONS

This application claims priority from PCT Application No. PCT/US2018/054042 filed on Oct. 2, 2018, which claims priority from U.S. Provisional Application Serial No. 62/567,096 filed on October 2, 2017, both of which are incorporated herein by reference in their entirety.

### STATEMENT REGARDING FEDERALLY SPONSORED RESEARCH OR DEVELOPMENT

Not Applicable.

### FIELD

The field of the invention relates to proliferative diseases and to biomarkers of response to immunotherapy, including pharmaceutical agents and antibodies used for the prevention and the treatment of cancer.

### INTRODUCTION

Definition of suitable biomarkers of response to treatment can highly impact disease outcome and progression. In oncology particularly, there is a need for highly specific and sensitive prognostic and predictive markers. The information relative to these biomarkers can be obtained from a tumor biopsy, later analyzed using molecular methods including but not limited to genomics and sequencing, transcriptomics, proteomics.

Immunotherapy agents are drugs that harness and enhance the capacity of the innate immune system to fight proliferative diseases. Indeed, cancer immunotherapy has been proven efficient even for tumors resistant to chemotherapy and radiation therapy, and thus offer the possibility for a long-term cancer remission. Multiple biomarkers of response to immunotherapies have been developed, but there is yet no comparison, standardization or prospective validation of these companion assays. Expression of proteins directly targeted by such agents on tumor cells and/or tumor-infiltrating lymphocytes (e.g. Programmed-cell Death Ligand 1 (PD-L1) protein staining for PD-1/PD-L1 axis inhibitors) only constitutes a part of the predictive model for the response to the drugs, and additional biomarkers are needed. Various embodiments of the invention described below meet this need as well as other needs existing in the field of diagnosing and treating cancer.

The immune system exhibits ubiquitous properties, such as context-dependent response to pathogens and non-self-elements, continuous learning, and memory. The overall mechanisms behind these features seem rather common between individuals and populations. Recently, it has been hypothesized that the variability of response to cancer immunotherapy mostly depends on the intrinsic heterogeneity of the tumor, largely highlighted by the uniqueness of one's tumor mutation profile. This molecular ‘fingerprint’ may be reflected by a unique neo-antigens catalog, presenting a certain level of “non-selfness”, further eliciting or repressing the immune response.

The present invention provides a method to estimate the antigenicity (i.e. the probability for a peptide to be presented by the major histocompatibility complex (MHC) to the immune system) and/or immunogenicity (i.e. the probability for a peptide to be recognized by the immune system) of the set of neo-peptides presented by one tumor, given its specific mutation description. This method comprises: (i) describing the unique set of DNA or RNA mutations presented by a tumor sample; (ii) determining the set of all possible 8- to 10-mers neo-epitopes encoded by the nucleic acid or protein sequences encompassing the mutations observed; (iii) defining the physicochemical properties of the set of neo-epitopes produced by the tumor cell, particularly their overall hydrophobicity and specific amino-acid content; (iv) assessing the antigenicity and immunogenicity of the set of neo-epitopes; and (v) estimating the further patient's response to immunotherapies, based on the set of neo-epitopes actually presented by the tumor cells to the immune system.

### SUMMARY

The present teachings include methods for prediction of response to immunotherapy for patients diagnosed with a proliferative, degenerative or inflammatory disease, by analysis of physicochemical properties of the set of neo-antigens produced by the injured tissue, comprising description of genomic or/and protein alterations in a sample. The set of alterations described may be obtained by a validated assay that involves: a) contacting the sample with one or more agents that detect genomic and/or protein variations in at least one molecular marker; b) comparing the sequence(s) of at least one genomic or protein marker detected in the sample with this of a reference genome or a reference proteome; and c) defining a list of genomic or protein alterations specific to the sample; elucidation of all possible peptides encompassing the genomic and/or protein alterations observed in the tumor; description of the physicochemical properties of the set of neo-epitopes possibly produced by the tumor cell, as compared to the epitopes normally presented by a healthy/non-mutated cell; estimation of the antigenicity and immunogenicity of the set of neo-epitopes, based on the physicochemical properties of these antigens; use of the antigenicity and immunogenicity estimates as biomarkers for prediction of the patient's response to immunotherapy.

In an aspect, the sample is obtained from a cancer patient. The sample may be a tumor biopsy, or a body fluid containing tumor biomolecules. In various aspects, the molecular alterations are missense, non-sense, non-stop, small deletions, small insertions, or frameshift mutations. The alterations observed can be specifically related to an endogenous mutagenesis mechanism. In another aspect, the endogenous mechanism underlying the mutations observed in the tumor sample can be caused by the cytidine-deaminase AID/APOBEC family of enzymes.

In various embodiments, the endogenous mechanism underlying the mutations observed in the tumor sample respect the nucleotide patterns TCW→TKW or WGA→WMA where T represents a thymine, C represents a cytosine, G represents a guanine, A represents an adenine, W represents an A or a T, K represents a G or T, and M represents an A or C. In various embodiments, the alterations observed are specifically related to an exogenous mutagenesis mechanism. In various embodiments, the exogenous mechanism underlying the mutations observed in the tumor sample is caused by exposure to ultra-violet (UV) radiation.

In various embodiments, the exogenous mechanism underlying the mutations observed in the tumor sample respect the nucleotide pattern TCC→TTC or GGA→GAA where T represents a thymine, C represents a cytosine, G represents a guanine, A represents an adenine. In various embodiments, the size of the peptides allow their presentation by the major histocompatibility complex (MHC) class I. In various embodiments, the definition of peptides includes the retrieval of all 8 amino-acids contiguous from both sides to the alterations detected.

In various embodiments, the alterations detected can be located at position 1 to 8 within said peptides.

In yet other aspects of the present invention, peptides are provided having the formula XᵢXᵢXᵢXᵢXᵢXᵢXₘ, XᵢXᵢXᵢXᵢXᵢXₘXₘ, XᵢXᵢXᵢXᵢXᵢXₘXₘXₘ, XᵢXᵢXᵢXᵢXₘXₘXₘXₘ, XᵢXᵢXᵢXₘXₘXₘXₘXₘ, XᵢXᵢXₘXₘXₘXₘXₘXₘ, XᵢXₘXₘXₘXₘXₘXₘXₘ or XₘXₘXₘXₘXₘXₘXₘXₘ wherein Xᵢ corresponds to the amino-acid(s) considered conserved (i.e. not different from the reference); and Xₘ corresponds to the amino-acid(s) altered or potentially altered by the mutation observed in the marker of interest.

In various embodiments, the definition of peptides includes the retrieval of all 9 amino-acids contiguous to the alterations detected. In various embodiments, the alterations detected can be located at position 1 to 9 within said peptides.

In yet other aspects of the present invention, peptides are provided having the formula XᵢXᵢXᵢXᵢXᵢXᵢXᵢXₘ, XᵢXᵢXᵢXᵢXᵢXᵢXᵢXₘXₘ, XᵢXᵢXᵢXᵢXᵢXᵢXₘXₘXₘ, XᵢXᵢXᵢXᵢXᵢXₘXₘXₘXₘ, XᵢXᵢXᵢXᵢXₘXₘXₘXₘXₘ, XᵢXᵢXᵢXₘXₘXₘXₘXₘXₘ, XᵢXₘXₘXₘXₘXₘXₘXₘ, XᵢXₘXₘXₘXₘXₘXₘXₘXₘ or XₘXₘXₘXₘXₘXₘXₘXₘXₘ wherein Xᵢ corresponds to the amino-acid(s) considered conserved (i.e. not different from the reference); and Xₘ corresponds to the amino-acid(s) altered or potentially altered by the mutation observed in the marker of interest.

In various embodiments, the definition of peptides includes the retrieval of all 10 amino-acids contiguous from both sides to the alterations detected. In various embodiments, the alterations detected can be located at position 1 to 10 within said peptides.

In yet other aspects of the present invention, peptides are provided having the formula XᵢXᵢXᵢXᵢXᵢXᵢXᵢXᵢXₘ, XᵢXᵢXᵢXᵢXᵢXᵢXᵢXᵢXₘXₘ, XᵢXᵢXᵢXᵢXᵢXᵢXᵢXₘXₘXₘ, XᵢXᵢXᵢXᵢXᵢXᵢXₘXₘXₘXₘ, XᵢXᵢXᵢXᵢXᵢXₘXₘXₘXₘXₘ, XᵢXᵢXᵢXᵢXₘXₘXₘXₘXₘXₘ, XᵢXᵢXₘXₘXₘXₘXₘXₘXₘ, XᵢXₘXₘXₘXₘXₘXₘXₘXₘ, XᵢXₘXₘXₘXₘXₘXₘXₘXₘXₘ or XₘXₘXₘXₘXₘXₘXₘXₘXₘXₘ wherein Xᵢ corresponds to the amino-acid(s) considered conserved (i.e. not different from the reference); and Xₘ corresponds to the amino-acid(s) altered or potentially altered by the mutation observed in the marker of interest. In various embodiments, the physicochemical properties of each epitope include hydrophobicity, amino-acid content, size, charge, polarity, amino-acid side-chain bonds, tertiary conformation and steric parameters. In various embodiments, the neo-epitopes produced by the tumor cell present an increase of hydrophobicity compared to the non-mutated epitopes. In various embodiments, the neo-epitopes produced by the tumor cell present an increase of valine (V, Val) or/and isoleucine (Ile, I) or/and leucine (Leu, L), methionine (Met, M) or/and phenylalanine (Phe, F) or/and alanine (Ala, A) or/and cysteine (Cys, C) amino-acid content compared to the non-mutated epitopes. In various embodiments, the antigenicity of one neo-epitope is dependent of its binding to the MHC class I moieties.

In various embodiments, one neo-epitope may be presented by the MHC class I isotypes HLA-A, HLA-B, HLA-C, HLA-E, HLA-F, HLA-G, HLA-K or HLA-L. In various embodiments, the binding to the MHC class I moieties is proportional to the neo-epitope hydrophobicity. In various embodiments, the hydrophobicity of one neo-epitope is determined by summing the hydrophobicity of each amino-acid included in said peptide. In various embodiments, the hydrophobicity of the complete set of tumor neo-epitopes is determined by summing the hydrophobicity corresponding to each peptide observed. In various embodiments, the immunogenicity of one neo-epitope is dependent of its recognition by a specific immune-cell receptor. In various embodiments, the immune-cell receptor is the T-cell receptor (TCR) located at the surface of the cytotoxic T lymphocytes. In various embodiments, the recognition by the immune-cell receptor is predicted to be proportional to the neo-epitope hydrophobicity. In various embodiments, the hydrophobicity of one neo-epitope is determined by summing the hydrophobicity of each amino-acid included in said peptide. In various embodiments, the hydrophobicity of the complete set of tumor neo-epitopes is determined by summing the hydrophobicity corresponding to each peptide observed.

In various embodiments, the patient is treated by checkpoint inhibitor. In various embodiments, the patient's response to immunotherapy is directly proportional to the mutational pattern retrieved from the teachings herein. In various embodiments, the patient's response to immunotherapy is directly proportional to the mutational pattern caused by the AID/APOBEC family of enzymes. In various embodiments, the patient's response to immunotherapy is directly proportional to the mutational pattern caused by an exposure to UV radiation. In various embodiments, the tumor-specific expression of immune checkpoints is proportional to the mutational pattern retrieved from the teachings herein. In various embodiments, the immune checkpoints considered are PD-L1, PD-L2, PD-1, CTLA-4 or BTLA.

In various embodiments, the immune checkpoint expression is proportional to the mutational pattern caused by the AID/APOBEC family of enzymes. In various embodiments, the immune checkpoint expression is proportional to the mutational pattern caused by an exposure to UV radiation. In various embodiments, the patient's predicted response to immunotherapy is directly proportional to the neo-epitope physicochemical properties retrieved from the teachings herein. In various embodiments, the patient's predicted response to immunotherapy is directly proportional to the increase of hydrophobicity of the neo-epitopes produced by the tumor, compared to the non-mutated epitopes. In various embodiments, the patient's predicted response to immunotherapy is directly proportional to the increase of valine (V, Val) or/and isoleucine (Ile, I), or/and leucine (Leu, L) or/and methionine (Met, M) or/and phenylalanine (Phe, F) or/and alanine (Ala, A) or/and cysteine (Cys, C) amino-acid content of the neo-epitopes produced by the tumor, compared to the non-mutated epitopes. In various embodiments, the tumor-specific expression of immune checkpoints is predicted to be proportional to the neo-epitope physicochemical properties retrieved from the teachings herein.

In various embodiments, the immune checkpoints considered are PD-L1, PD-L2, PD-1, CTLA-4 or BTLA. In various embodiments, the immune checkpoint expression is predicted to be proportional to the increase of hydrophobicity of the neo-epitopes produced by the tumor, compared to the non-mutated epitopes. In various embodiments, the immune checkpoint expression is predicted to be proportional to the increase of valine (V, Val) or/and isoleucine (Ile, I) or/and leucine (Leu, L) or/and methionine (Met, M) or/and phenylalanine (Phe, F) or/and alanine (Ala, A) or/and cysteine (Cys, C) amino-acid content of the neo-epitopes produced by the tumor, compared to the non-mutated epitopes.

These and other features, aspects and advantages of the present teachings will become better understood with reference to the following description, examples and appended claims.

### DRAWINGS

Those of skill in the art will understand that the drawings, described below, are for illustrative purposes only. The drawings are not intended to limit the scope of the present teachings in any way.

FIG. 1. Overall hydrophobicity change of the human coding genome, after multiple iterations of kataegis or UV exposure (computed in silico−N=1 to 100 iterations).

FIG. 2. Cumulative change in hydrophobicity of 8- to 10-mer neo-antigens in human tumor samples and correlation with

### DETAILED DESCRIPTION

### EXAMPLES

### Example 1

AID/APOBEC mutational signature is associated with an increase of neo-peptide hydrophobicity and PD-L1 mRNA expression in a large collection of human tumor samples.

To illustrate the methods described above, we downloaded the molecular profile (point mutations and small insertions/deletions and mRNA expression data obtained by next-generation sequencing (NGS) methods) of 469 highly-mutated pan-cancer human tumors, available without restriction of use from the community resource project The Cancer Genome Atlas (TCGA) (Broad GDAC Firehose website: https://gdac.broadinstitute.org—standardized data run release 2016 01 28. All samples were published and available on the date of Mar. 1, 2017), and for which the presence of an AID/APOBEC mutational signature was previously determined by the P-MACD (Pattern of Mutagenesis by APOBEC Cytidine Deaminases analysis) computation method (Roberts, S. A. et al., An APOBEC cytidine deaminase mutagenesis pattern is widespread in human cancers, Nature Genetics 45:970-976 (2013)).

Using the mutation description available for these tumors, we generated all possible 8-mer to 10-mer neo-peptides encompassing each mutation (n=2,660,232 epitopes located in 15,163 different gene products). The differences in total hydrophobicity (i.e. the sum of hydrophobicity of all residues) of the neo-peptides after versus before mutagenesis was then considered. The results obtained were computed in two ways—either not weighted by mRNA expression levels or weighted by these levels (in order to take into consideration whether the neo-antigens were actually transcribed and their respective levels of expression). Finally, the hydrophobicity and expression of immune markers of tumors harboring an AID/APOBEC mutational signature were compared to those without, using a Wilcoxon-Mann-Whitney rank-sum test and a Fisher's exact test, respectively.

Here, we showed that highly mutated tumors (top 30% tumor mutation burden in the TCGA database) presenting an AID/APOBEC mutational signature presented a significant increase in terms of overall change in hydrophobicity in comparison to tumors not altered by the AID/APOBEC enzymes (mean [confidence interval 95% (CI₉₅%)] =8,702 [7,506-9,898] versus 3,374 [2,987-3,761] arbitrary units (AU)−p-value <0.0001−Table 1). This difference remained significant when the change in hydrophobicity score was weighted by the expression level of each transcript (mean [CI₉₅%] =22.2 [17.7−26.6]×10⁸versus 2.6 [−8.9−14.2]×10⁸ AU−p-value <0.0001−Table 1).

TABLE 1
Comparison of change in hydrophobicity score of the
neo-peptide library (8- to 10-mer peptides) of TCGA
tumors with and without AID/APOBEC mutagenesis.

| | Top 30% of tumors by mutational burden (n = 469) | | | | |
|---|---|---|---|---|---|
| | Tumors without | | Tumors with | | |
| | AID/APOBEC | | AID/APOBEC | | |
| | signature (n = 239) | | signature (n = 230) | | p-value |
| | Change in hydrophobicity score by tumor | | | | |
| Mean | 3,374 | [2,987-3,761] | 8,702 | [7,506-9,898] | <0.0001 |
| [CI, | | | | | |
| 95%] | | | | | |
| Median | 2,763 | [−1,692-22,428] | 5,587 | [765-70,444] | |
| [range] | | | | | |
| | Weighted change in hydrophobicity score, by tumor | | | | |
| Mean | 2.6 | [−8.9-14.2] × 10⁸ | 22.2 | [17.7-26.6] × 10⁸ | <0.0001 |
| [CI, | | | | | |
| 95%] | | | | | |
| Median | 5.1 | [−1,344-215] × 10⁸ | 11.5 | [−63-291] × 10⁸ | |
| [range] | | | | | |

Abbreviation: CI, 95% = 95% confidence interval

Interestingly, an extended analysis of the expression of common lymphocyte and monocyte markers between tumors presenting an AID/APOBEC mutational signature versus tumors not impacted by APOBEC hyper-activity (excluding melanoma) also revealed an association with the overexpression of the PD-L1 and/or PD-L2 ligands (Odds Ratio (OR) =4.20, p-value =0.0023). The expression of interferon gamma (IFNγ), a marker of lymphocyte activation, was found significantly and similarly associated with the presence of an AID/APOBEC mutational signature (p-value=0.0023). Additionally, T-cell specific markers, such as CD4 (associated with the presence of CD4+ helper T cells) and CD8A (associated with the presence of CD8+ cytotoxic T cells), were significantly and positively associated with the AID/APOBEC mutational signature (OR=3.4 and 4.3 respectively , p-values <0.0095) (Table 2).

TABLE 2
Immune response markers associated with the
presence of an AID/APOBEC mutational signature
in a set of human pan-cancer tumors.*

| Tumors | | | |
|---|---|---|---|
| presenting | | | |
| AID/APOBEC | | | |
| signature | | | Odd Ratio |
| Yes (%) | No | p-value | [CI_{95%}] |

High mutation burden tumors (n = 408)

| | | | | | |
|---|---|---|---|---|---|
| Presence of | 76.4% | 73.8% | 0.6942 | 1.15 | [0.69-1.92] |
| lymphocyte infiltrate | | | | | |
| Presence of monocyte | 30.0% | 42.0% | 0.0413 | 0.59 | [0.37-0.96] |
| infiltrate | | | | | |
| CD3G overexpression | 7.9% | 3.9% | 0.1281 | 2.10 | [0.89-4.96] |
| CD8A overexpression | 11.8% | 3.0% | 0.0006 | 4.26 | [1.77-10.27] |
| CD4 overexpression | 9.6% | 3.0% | 0.0095 | 3.36 | [1.36-8.30] |
| MS4A1 | 4.5% | 2.2% | 0.2562 | 2.12 | [0.68-6.59] |
| overexpression | | | | | |
| CD14 overexpression | 7.3% | 4.8% | 0.2967 | 1.57 | [0.69-3.59] |
| CD33 overexpression | 6.7% | 2.6% | 0.0527 | 2.70 | [0.99-7.34] |
| IL3RA overexpression | 6.2% | 5.2% | 0.6725 | 1.20 | [0.52-2.78] |
| NCAM1 | 0.6% | 1.7% | 0.3923 | 0.32 | [0.04-2.88] |
| overexpression | | | | | |
| IFNG overexpression | 10.1% | 2.6% | 0.0023 | 4.20 | [1.63-10.82] |
| PD-L1/2 | 10.1% | 2.6% | 0.0023 | 4.20 | [1.63-10.82] |
| overexpression | | | | | |

### Example 2

AID/APOBEC mutational signature is associated with a better outcome following treatment by PD-1/PD-L1 blockade.

In this example, we aimed at studying if, whether or not, the tumor AID/APOBEC mutational signature is associated with a higher response to immunotherapy. A cohort of 99 patients (including 36 with non-small cell lung cancers and 63 with diverse advanced cancers other than melanoma) previously treated by immunotherapy revealed that the response to immunotherapy is associated with the ‘AID/APOBEC high mutation status’; patients with a high APOBEC status were more likely to have a complete (CR) or partial (PR) response (OR=9.69, p-value 0.0106). Additionally, patients with a high APOBEC status had a median PFS of 3.1 months while those with low APOBEC had a median PFS of only 2.1 months (p-value=0.0239) (Table 3).

TABLE 3
APOBEC mutational status of 99 pan-cancer
tumors and response to immunotherapy.

| | | High or | | Low or | | |
|---|---|---|---|---|---|---|
| | | positive | | negative | | |
| | | APOBEC | | APOBEC | | |
| | | signature | | signature | | |
| Variable | Group | N = 70 (71%) | | N = 29 (29%) | | P-value |
| Clinical | CR/PR | 18 | (26%) | 1 | (3%) | 0.0106 |
| response | SD or PD | 52 | (74%) | 28 | (97%) | OR = 9.69 |
| | | | | | | (95% CI |
| | | | | | | 1.46-104.8) |
| PFS | Median | 3.1 | (0.2-22.4+) | 2.1 | (0.4-15.9) | 0.0239 |
| (range) | (months) | | | | | HR = 0.60 |
| | | | | | | (95% CI |
| | | | | | | 0.37-0.99) |

Abbreviations: CI = confidence interval; CR = complete response; HR = hazard ratio; OR = odds ratio; PD-1 = programmed death receptor-1; PD = progressive disease: PFS = progression free survival; PR = partial response; SD = stable disease.

### Example 3

AID/APOBEC and UV mutational signatures induce an increase of neo-peptide hydrophobicity, as revealed by an in silico computation and analysis of repository pan-cancer human samples.

All possible 6-nucleotides stretches (n=4,096) observed in the human coding genome were used as a template for in silico mutagenesis analysis. The nucleotide pattern description of AID/APOBEC signature described by Alexandrov et al. (Alexandrov L B, Nik-Zainal S, Wedge D C, Aparicio SAJR, Behjati S, Biankin A V, et al. Signatures of mutational processes in human cancer. Nature. 22 aoilt 2013; 500(7463):415-21) was applied on this set of virtual stretches. Overall, 192 virtual single-nucleotide substitutions caused by AID/APOBEC enzymes were applied in silico on the set of stretches, resulting in a total of 786,432 possible changes. The difference in total hydrophobicity corresponding to each nucleotide stretch (i.e. the hydrophobicity of possible peptides resulting from these virtual stretches) before and after single-round of APOBEC mutagenesis was then evaluated using a Wilcoxon signed-rank test (Table 4). Application of our in silico mutagenesis method resulted in a significant difference of hydrophobicity ranks for stretches presenting a kataegis mutation (n=3,744 (91.4% of existing stretches)−p-value <0.0001). The median hydrophobicity change per stretch was positive (median=+1.0×10-7 arbitrary unit (AU)), and the sum of all hydrophobicity changes—corresponding to the hydrophobicity change observed after creation of a single APOBEC alteration in the complete human coding genome, weighted by the probability that the mutation occurs within a given stretch, was equal to +0.0235 AU.

TABLE 4
Consequences of a single iteration of APOBEC mutagenesis
on the overall hydrophobicity of the human coding
genome (per in silico computation).

| | HYDROPHOBICITY SCORE | | |
|---|---|---|---|
| | Before | After | |
| | kataegis | kataegis | Difference |
| Number of mutated | 3744 | | |
| stretches | | | |
| Median | 0 | −0.000003384 | +1.0 × 10⁻⁷ |
| 25% percentile | −0.000486 | −0.0004733 | −1.3 × 10⁻⁷ |
| 75% percentile | 0.0004366 | 0.000448 | 1.3 × 10⁻⁶ |
| Mean | −0.000009969 | −0.000003683 | +6.3 × 10⁻⁶ |
| Standard deviation | 0.001202 | 0.001199 | 2.2 × 10⁻⁵ |
| Standard error | 0.00001964 | 0.00001959 | 3.5 × 10⁻⁷ |
| Lower 95% CI | −0.00004847 | −0.00004209 | 5.6 × 10⁻⁶ |
| Upper 95% CI | 0.00002853 | 0.00003472 | 7.0 × 10⁻⁶ |
| Sum | −0.03732 | −0.01379 | +0.0235 |

| | |
|---|---|
| P-value | <0.0001 |
| Wilcoxon signed | |
| rank test | |

Abbreviations: CI = confidence interval.

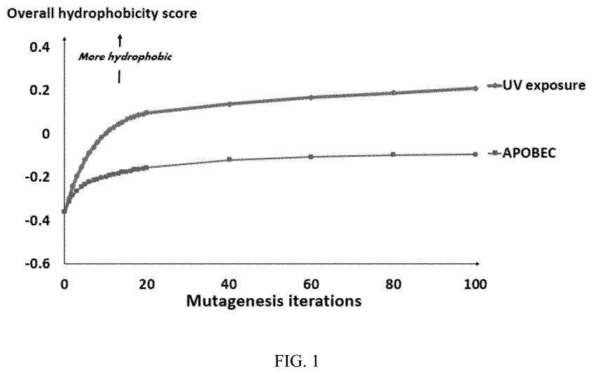

With the intention to mimic the effect of the APOBEC hyper-activity observed in human tumors (TCGA samples present an average of 60 kataegis-related mutations per tumor) or the regular exposure to UV, we evaluated the impact of repeated in silico mutagenesis over the estimated hydrophobicity of the complete human coding genome (the mutated stretches being used as template for additional rounds of mutagenesis). As shown in FIG. 1, the reference (baseline) coding genome tends to be hydrophilic, with a score of -0.36 AU (calculated by summing the scores of all 6-nucleotide stretches, weighted by frequencies of observation within the genome). After 100 rounds of kataegis, the overall hydrophobicity was estimated at −0.09 AU, which corresponds to an increase of hydrophobicity of +75% (+0.27 AU). After 100 rounds of UV-related mutagenesis, the overall hydrophobicity was estimated at +0.21 AU, which corresponds to an increase of hydrophobicity of +158% (+0.57 AU) (FIG. 1).

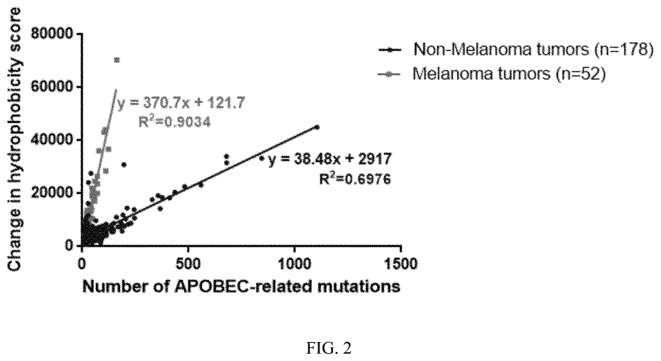

These results were later confirmed on a set of highly-mutated tumors (TCGA database, n=469 tumor samples). Mutation descriptions for each tumor were used to generate 8- to 10-mers neo-antigens pools. A total of 2,660,232 neo-antigens was computed. The change in hydrophobicity of these neo-antigens before and after mutagenesis (as compared to the human reference genome GRCh37) was then summed by tumor, and plotted against the number of APOBEC-related mutations within each associated tumor (FIG. 2). The correlation between the overall neo-antigen hydrophobicity and the number of APOBEC-related mutation was significant (p<0.0001), but with a low coefficient (R²=0.2741) and the graph presented in a ‘fish-tail’ shape. This shape allowed discrimination of 2 groups of tumors: melanoma (n=52) and non-melanoma (n=178) samples. Correlation coefficients for these groups considered separately were R²=0.9034 for melanoma and R²=0.6976 for non-melanoma tumors. Both correlations presented a p-value <0.0001 (FIG. 2). Interestingly, prevalence of melanoma tumors is highly associated with UV exposure, and therefore the 2 groups presented in the graph separate tumors presenting an UV-mutation signature from tumors presenting an APOBEC-mutation signature.

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A method of prediction of response to immunotherapy for patients diagnosed with a proliferative, degenerative or inflammatory disease, by analysis of physicochemical properties of the set of neo-antigens produced by the injured tissue, comprising:
obtaining a description of genomic or/and protein alterations in a sample, wherein the set of alterations are obtained by validated assay that involves: a) contacting the sample with one or more agents that detect genomic and/or protein variations in at least one molecular marker; b) comparing sequences of at least one genomic or protein marker detected in the sample with a reference genome or a reference proteome; and c) defining a list of genomic or protein alterations specific to the sample, wherein the sample is tumor biopsy or a body fluid containing tumor biomolecules obtained from a cancer patient;
elucidating possible peptides encompassing the genomic and/or protein alterations observed in the tumor;
comparing a description of the physicochemical properties of the set of neo-epitopes possibly produced by the tumor cell to epitopes normally presented by a healthy/non-mutated cell;
estimating antigenicity and immunogenicity of the set of neo-epitopes, based on the physicochemical properties of these antigens; and
using the antigenicity and immunogenicity estimates as biomarkers for prediction of the patient's response to immunotherapy
wherein the physicochemical properties of each epitope include hydrophobicity, amino-acid content, size, charge, polarity, amino-acid side-chain bonds, tertiary conformation and steric parameters

2. (canceled)

3. (canceled)

4. (canceled)

5. The method of claim 1, wherein the molecular alterations are: (i) missense, non-sense, non-stop, small deletions, small insertions, or frameshift mutations; (ii) observed related to an endogenous mutagenesis mechanism, wherein the endogenous mechanism is underlying the mutations observed in the tumor sample caused by the cytidine-deaminase AID/APOBEC family of enzymes; and (iii) observed are specifically related to an exogenous mutagenesis mechanism.

6. (canceled)

7. (canceled)

8. The method of claim 1, wherein the endogenous mechanism underlying the mutations observed in the tumor sample respect the nucleotide patterns TCW→TKW or WGA→WMA, where T represents a thymine, C represents a cytosine, G represents a guanine, A represents an adenine, W represents an A or a T, K represents a G or T, and M represents an A or C.

9. (canceled)

10. The method of claim 1, wherein the exogenous mechanism underlying the mutations observed in the tumor sample is caused by exposure to ultra-violet (UV) radiation.

11. The method of claim 1, wherein the exogenous mechanism underlying the mutations observed in the tumor sample respect the nucleotide pattern TCW→TKW or WGA→WMA, where T represents a thymine, C represents a cytosine, G represents a guanine, A represents an adenine.

12. The method of claim 1, wherein the peptides comprises a size of the peptides allowing for presentation by histocompatibility complex (MHC) class I; a definition for retrieval of 8 amino-acids contiguous from both sides to the alterations detected, alterations detected at position 1 to 8 within the pepitides, a definition of peptides includes the retrieval of all 9 amino-acids contiguous to the alterations detected, alterations detected can be located at position 1 to 9 within said peptides, a definition of peptides includes the retrieval of all 10 amino-acids contiguous from both sides to the alterations detected, alterations detected can be located at position 1 to 10 within said peptides, wherein the MHC class I comprise moieties binding dependent on antigenicity of one neo-epitope.

13. (canceled)

14. (canceled)

15. The peptides of claim 1 having the formula XᵢXᵢXᵢXᵢXᵢXᵢXᵢXₘ, XᵢXᵢXᵢXᵢXᵢXᵢXₘX_{iM} XᵢXᵢXᵢXᵢXᵢXₘXₘX_{iM} XᵢXᵢXᵢXᵢXₘXₘXₘXₘ, XᵢXᵢXᵢXₘXₘXₘXₘXₘ, XᵢXᵢXₘXₘXₘXₘXₘXₘ, XᵢXₘXₘXₘXₘXₘXₘXₘ or XₘXₘXₘXₘXₘXₘXₘXₘ wherein Xᵢ corresponds to the amino-acid(s) considered conserved and Xₘ corresponds to the amino-acid(s) altered or potentially altered by the mutation observed in the marker of interest.

16. (canceled)

17. (canceled)

18. The peptides of claim 1 having the formula XᵢXᵢXᵢXᵢXᵢXᵢXᵢXᵢXₘ, XᵢXᵢXᵢXᵢXᵢXᵢXᵢXₘXₘ, XᵢXᵢXᵢXᵢXᵢXᵢXₘXₘXₘ, XᵢXᵢXᵢXᵢXᵢXₘXₘXₘXₘ, XᵢXᵢXᵢXᵢXₘXₘXₘXₘXₘ, XᵢXᵢXᵢXₘXₘXₘXₘXₘXₘ, XᵢXᵢXₘXₘXₘXₘXₘXₘXₘ, XᵢXₘXₘXₘXₘXₘXₘXₘXₘ or XₘXₘXₘXₘXₘXₘXₘXₘXₘ wherein Xᵢ corresponds to the amino-acid(s) considered conserved; and Xₘ corresponds to the amino-acid(s) altered or potentially altered by the mutation observed in the marker of interest.

19. (canceled)

20. (canceled)

21. The peptides of claim 1 having the formula XᵢXᵢXᵢXᵢXᵢXᵢXᵢXᵢXᵢXₘ, XᵢXᵢXᵢXᵢXᵢXᵢXᵢXᵢXₘXₘ, XᵢXᵢXᵢXᵢXᵢXᵢXᵢXₘXₘXₘ, XᵢXᵢXᵢXᵢXᵢXᵢXₘXₘXₘXₘ, XᵢXᵢXᵢXᵢXᵢXₘXₘXₘXₘXₘ, XᵢXᵢXᵢXᵢXₘXₘXₘXₘXₘXₘ, XᵢXᵢXₘXₘXₘXₘXₘXₘXₘ XᵢXₘXₘXₘXₘXₘXₘXₘXₘ XᵢXₘXₘXₘXₘXₘXₘXₘXₘXₘ or XₘXₘXₘXₘXₘXₘXₘXₘXₘXₘ wherein Xᵢ corresponds to the amino-acid(s) considered conserved and Xₘ corresponds to the amino-acid(s) altered or potentially altered by the mutation observed in the marker of interest.

22. (canceled)

23. The method of claim 1, wherein the neo-epitopes produced by the tumor cell present an increase of hydrophobicity compared to the non-mutated epitopesᵢ an increase of valine (V, Val) or/and isoleucine (Ile, I) or/and leucine (Leu, L), methionine (Met, M) or/and phenylalanine (Phe, F) or/and alanine (Ala, A) or/and cysteine (Cys, C) amino-acid content compared to the non-mutated epitopes.

24. (canceled)

25. (canceled)

26. The method of claim 1, wherein one neo-epitope is presented by the MEW class I isotypes HLA-A, HLA-B, HLA-C, HLA-E, HLA-F, HLA-G, HLA-K or HLA-L.

27. The method of claim 26, wherein the binding to the MHC class I moieties is proportional to the neo-epitope hydrophobicity.

28. The method of claim 1, further comprising: the hydrophobicity of one neo-epitope determined by summing the hydrophobicity of each amino-acid included in said peptide and used to predict the recognition by the immune-cell receptor; the hydrophobicity of the complete set of tumor neo-epitopes determined by summing the hydrophobicity corresponding to each peptide observed; the hydrophobicity of one neo-epitope determined by summing the hydrophobicity of each amino-acid included in said peptide; the hydrophobicity of the complete set of tumor neo-epitopes determined by summing the hydrophobicity corresponding to each peptide observed.

29. (canceled)

30. The method of claim 1, wherein the immunogenicity of one neo-epitope is dependent on recognition by a specific immune-cell receptor.

31. The method of claim 1, wherein the immune-cell receptor is the T-cell receptor (TCR) located at the surface of the cytotoxic T lymphocytes.

32. (canceled)

33. (canceled)

34. (canceled)

35. The method of claim 1, wherein the patient is treated by checkpoint inhibitor and patient has a response to immunotherapy directly proportional to the mutational pattern retrieved, the mutational pattern caused by the AID/APOBEC family of enzymes, and the mutational pattern caused by an exposure to UV radiation.

36. (canceled)

37. (canceled)

38. (canceled) T

39. The method of claim 1, wherein the tumor-specific expression of immune checkpoints is proportional to the mutational pattern retrieved and predicted to be proportional to the neo-epitope physicochemical properties, whereby the patient's predicted response to immunotherapy is directly proportional to: (i) the increase of hydrophobicity of the neo-epitopes produced by the tumor in comparison to the non-mutated epitopes or (ii) the increase of valine (V, Val) or/and isoleucine (Ile, I), or/and leucine (Leu, L) or/and methionine (Met, M) or/and phenylalanine (Phe, F) or/and alanine (Ala, A) or/and cysteine (Cys, C) amino-acid content of the neo-epitopes produced by the tumor, compared to the non-mutated epitopes.

40. The method of claim 1, wherein the immune checkpoints considered are PD-L1, PD-L2, PD-1, CTLA-4 or BTLA.

41. The method of claim 1, wherein the immune checkpoint expression is proportional to the mutational pattern caused by the AID/APOBEC family of enzymes or the mutational pattern caused by an exposure to UV radiation.

42. (canceled)

43. (canceled)

44. (canceled)

45. (canceled)

46. (canceled)

47. (canceled)

48. The method of claim 1, wherein the immune checkpoint expression is predicted to be proportional to the increase of hydrophobicity of the neo-epitopes produced by the tumor, compared to the non-mutated epitopes or predicted to be proportional to the increase of valine (V, Val) or/and isoleucine (Ile, I) or/and leucine (Leu, L) or/and methionine (Met, M) or/and phenylalanine (Phe, F) or/and alanine (Ala, A) or/and cysteine (Cys, C) amino-acid content of the neo-epitopes produced by the tumor, compared to the non-mutated epitopes.

49. (canceled)
